(19) United States
(12) Patent Application Publication
(10) Pub. No.: US 2021/0161469 A1
(43) Pub. Date: Jun. 3, 2021

(54) A WEARABLE PROGNOSTIC DEVICE USED IN THE EARLY DIAGNOSIS OF BREAST CANCER AT HOME

(71) Applicant: Zeynep AKÇASIZ, Izmir (TR)

(72) Inventors: Bengi Alara AKÇASIZ, Izmir (TR); Noyan Alperen IDIN, Izmir (TR)

(21) Appl. No.: 16/972,457

(22) PCT Filed: Jun. 1, 2019
(86) PCT No.: PCT/TR2019/050417

§ 371 Date: Dec. 4, 2020

(30) Foreign Application Priority Data
Jun. 5, 2018 (TR) 2018/08005

(51) Int. Cl. A61B 5/00

(52) U.S. Cl. CPC A61B 5/6804; A61B 5/0053; A61B 2562/0215; A61B 2562/0247; A61B 5/4312

(57) ABSTRACT

A wearable textile product in the early diagnosis of the breast cancer which includes: a brassiere or a main body which covers the breast, support bars connected to its main body, a lock that is nestable or interlocking and connected to the support bars, swellable portions that are positioned according to the selection of the textile product with required size, form and location, a lower layer that is fixed to the main body of the product with interlocking form possessed by the swellable portions, an upper layer as a swellable flexible substance, a gas pump that is connected to the gas channel end holder of the portion that is swellable by means of gas transmission channels, a kit that detects the pressure within the swellable portions, a signal transmission channel, and a process control center.

### TECHNICAL FIELD

The invention relates to a product to be used in the early diagnosis of the breast cancer by alerting the patient in case of abnormality in breast tissue.

The invention is particularly related to a wearable product used in the early diagnosis of breast cancer and breast lumps at home.

### STATE OF THE ART

Today early diagnosis of the breast cancer is a very important and critical process in order to save the unhealthy area. The cancer that cannot be diagnosed early causes disappointing results.

The breast cancer diagnosis is made by means of palpation in the current art. The existence of the mass is tried to be determined by sensing this without using any auxiliary tool, equipment. However its validity is suspicious because it is not based on any technological data control system.

In a research made in the present state of the art, monogram device is found for the breast cancer diagnosis. These devices operate with radiation thus it is very dangerous for the women under 40 and for pregnant women. Also in order to use the device, a person skilled in the art or a doctor is required and it is not appropriate for using at home.

Again in a research made in the present state of the art, a breast mass diagnosis hand glove is seen. This product is worn to the hand and increases the feeling that we receive with our hands. It again does not provide a reliable result because particularly it leaves the diagnosis decision to a specialist.

Again an invention in the present state of the art numbered as “EP2591363B1” and titled as, “Breast Cancer Diagnosis and Treatment” and with classification number “G01N 33/574”, is related to the compositions and methods for cancer diagnosis, research and therapy including without any limitation cancer markers. The current invention is particularly related to compositions and methods for determining the response of a patient to cancer therapies.

Again, an invention numbered as “WO 1998/045328” and titled as “Compositions and methods for the diagnosis and treatment of the breast cancer” and with classification No “C07K 14/47”, discloses the compositions and methods for the diagnosis and therapy of the breast cancer. Among the provided compositions, there are preferably nucleotide sequences synthesized in the tumor tissue of the breast and also polypeptides that are coded by means of such type of nucleotide sequences. Vaccines and pharmaceutical compositions including such components are also provided and they can also be used for example for preventing and healing the breast cancer. The polypeptides are also used for the diagnosis of the patient with breast cancer and monitoring the progress of the disease and production of beneficial antibodies.

Once again an invention numbered as “TR 2015/17254” and titled as “A Training Kit for Self Breast Examination” and with classification No “G09B 23/28”, is related to a training kit which provides opportunity for training one person in an audial, visual and tactile manner in terms of self-examination in the breast cancer diagnosis (KKMM), comprises a monitor that is positioned on one main body, a silicone breast model, a counter button, a video player.

One again, an invention numbered as “TR 2012/02956” and titled as “Breast mass determination hand glove” and with classification No “A61B 19/04” is related to a hand glove in the health sector which helps mass diagnosis formed within the breasts of women. The product is a medical test tool to be used as a medical material, characterized in that; it is a breast mass determination hand glove for the early diagnosis of breast cancer findings with hand and determination of small masses around the breast.

### Aim of the Invention

In order to eliminate the disadvantages included within the present state of the art, the aim of the invention is a wearable device for providing alert for early diagnosis of breast cancer and following the changes in current breast lumps.

Another aim of the invention is that the mass detected by the hand, on the surface of breast, can be detected better by means of the sensors.

In order to provide the above mentioned advantages the invention is a brassiere product or a product that covers the breast to be used as a wearable textile product in the diagnosis of the breast cancer which comprises the following; a brassiere or a main body which covers the breast, support bars connected to its main body, a lock that is nestable or interlocking and connected to the support bars, swellable portions that are positioned according to the selection of the textile product with required size, form and location, a lower layer that is fixed to the main body of the product with interlocking form included by the swellable portions, an upper layer as a swellable flexible substance, a gas pump that is connected to the gas channel end holder of the portion that is swellable by means of gas transmission channels, a kit that detects the pressure within the swellable portions, signal transmission channel, process control center.

### DESCRIPTION OF FIGURES

The invention will be described with reference to the drawings attached hereto; therefore the characteristics of the invention will be clarified. However, the aim of this is not to limit the invention with specific embodiments. On the contrary, it is aimed to cover all alternatives, amendments and equivalents that comprise the described field of the claims of the invention attached hereto. The details shown are to be understood that they are only for describing the preferred embodiments of the present invention and also both for illustrating the methods and for providing the rules and conceptual features of the invention to be understood most appropriately and easily. In these figures;

FIG. 1 is a front open view of the inventive wearable textile product.

FIG. 2 is a rear open view of the inventive wearable textile product.

FIG. 3 is a view of the swellable portion of the invention.

FIG. 4 is a view of the pressure detection kit of the invention.

FIG. 5 is a view of the process control center.

FIG. 6 is a view of the pressure detection kit in an overlapping manner.

The figures that help clarifying this invention is enumerated as it is stated in the following figure and their names are given together with the numbers as the following.

Description of Reference Numbers

| Ref. No. | Description of the reference |
|---|---|
| 1 | Main body |
| 2 | Support bars |
| 3 | Lock |
| 4 | Gas pump |
| 5 | Gas transmission channel |
| 6 | Signal transmission channel |
| 7 | Process control center |
| 71 | Integrated multiplexer and demultiplexer |
| 72 | Communication module |
| 73 | Power source |
| 74 | Microprocessor |
| 8 | Swellable area |
| 81 | Lower layer of the swellable portion |
| 82 | Upper layer of the swellable portion |
| 9 | Fabric |
| 10 | Pressure detection kit |
| 101 | 1^{st} layer |
| 102 | 3^{rd} layer |
| 11 | Socket |
| 12 | Metal treads |
| 13 | Gas channel end holder |

### DESCRIPTION OF THE INVENTION

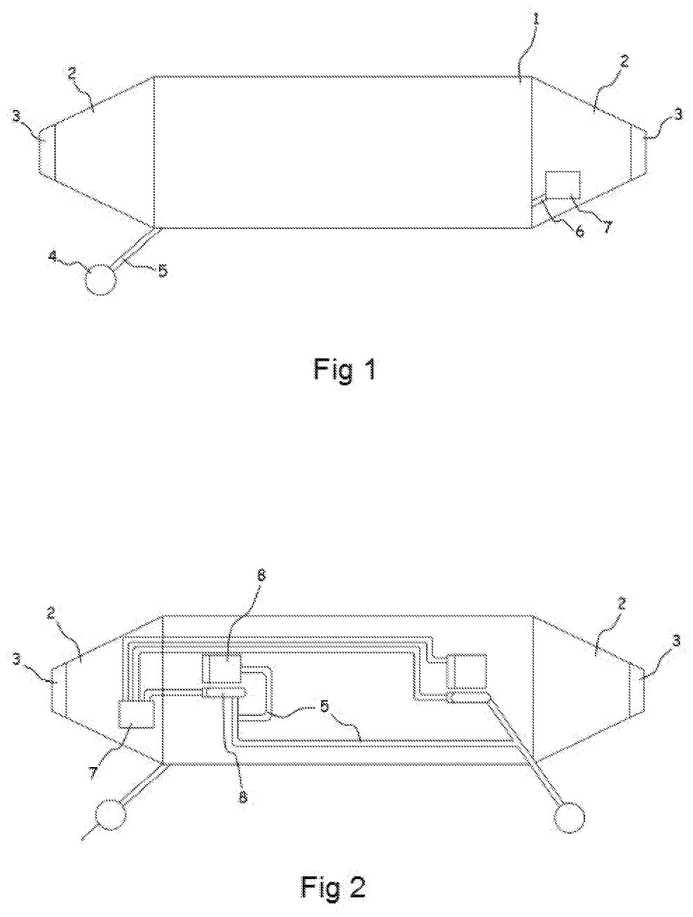

The inventive wearable textile product to be used for the diagnosis of the abnormal masses on the breast surface is a brassiere or a product that covers the breast. There are support bars (2) connected to the main body (1). A nestable or interlocking lock (3) or a similar apparatus is formed connected to the support bars (2) (FIG. 1).

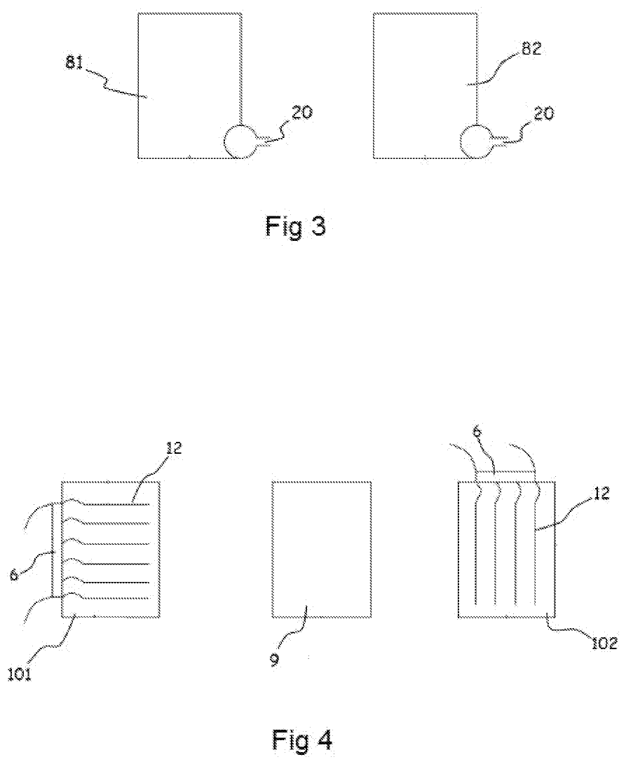

As it can be seen from FIG. 2, the swellable portions (8) are formed in a manner such that they can be located to various portions of the inventive wearable textile product. These swellable portions (8) can have any size, form or location. The swellable portion (8) consists of two layers. The lower layer (81) which is seen in FIG. 3 is fixed to the main body (1) of the product. The upper layer (82) which is also seen in FIG. 3 is a swellable flexible substance. The lower layer (81) and the upper layer (82) have an interlocking form.

There are gas transmission channels (5) which are seen in FIG. 2 and a gas pump (4) is located which is connected to the gas channel end holder (13) seen in FIG. 3 (FIG. 1). There is a pressure detection kit (10) within the swellable portions (8) of said product. A conductive fabric (9) with variable strength or an alternative other product can be used within the pressure detection kit (10).

The pressure detection kit (10) which is seen in FIG. 6 can consist of 3 or a variable number of layers. The 1^{st} layer (101) which is in FIG. 4 consists of horizontal strips formed with a variable number of silver or similar metal conductive treads (12). The 3^{rd} layer (102) consists of vertical strips formed with a variable number of silver or similar metal conductive treads (12). As it can be seen in FIG. 4, the interim 2^{nd} layer consists of a fabric (9) with variable strength or an alternative material. The conductive treads (12) are connected to the signal transmission channel (6). The signal transmission channel (6) is connected to the socket (11) in the process control center (7).

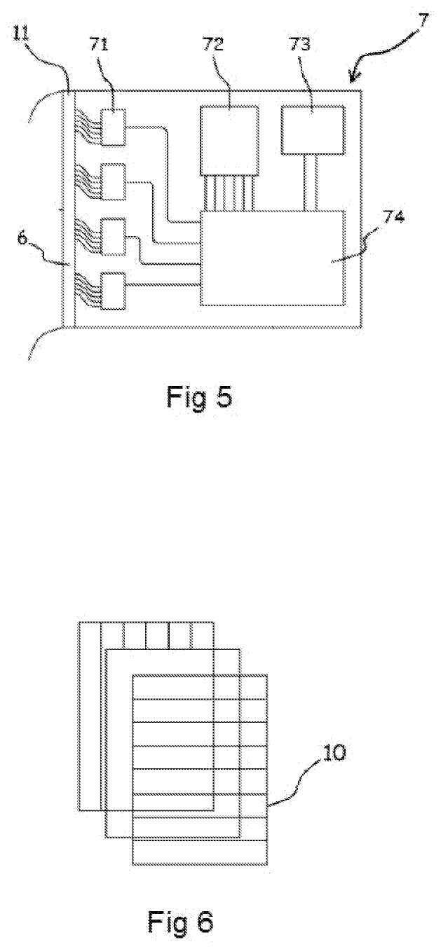

As it can be seen in FIG. 5, within the structure of the inventive cancer diagnosis product; there are a power source (73), integrated multiplexer and demultiplexer (71), microprocessor or signal driving circuit (74) and a process control center (7) with communication module (72).

1. A wearable textile product, to be used in the early diagnosis of the breast cancer, is a brassiere or a product that covers the breast, characterized in that; it comprises the following:
brassiere or a main body which covers the breast;
support bars connected to the main body;
a lock connected to the support bars, nestable or interlocking;
swellable portions of the textile product which have preferred size, form and location, possessing interlocking form included by said swellable portions;
a lower layer which is fixed to the main body of the product;
an upper layer as a swellable flexible material;
a gas pump located in a connected manner to the gas channel end holder of the swellable portion via gas transmission channels;
a pressure detection kit within the swellable portions;
a signal transmission channel; and
a process control center.

2. A product according to claim 1, wherein the pressure detection kit comprises a conductive fabric with variable strength.

3. A product according to claim 1, wherein said pressure detection kit comprises three or a variable number of layers.

4. A pressure detection kit according to claim 3, comprising a first layer comprising of horizontal strips formed with variable number of silver or similar metal conductive treads.

5. A pressure detection kit according to claim 3 comprising a third layer comprising of vertical strips formed with variable number of silver or similar metal conductive treads.

6. A pressure detection kit according to claim 3, comprising a 2nd layer comprising of a fabric with variable strength.

7. A product according to claim 1, comprising conductive treads connected to the signal transmission channel.

8. A product according to claim 1, wherein said signal transmission channel is connected to the socket in the process control center.

9. A product according to claim 1, wherein within the structure of said process control center, it comprises a power source, integrated multiplexer and demultiplexer, microprocessor or a signal driving circuit and a communication module.
